(19) United States
(12) Patent Application Publication
(10) Pub. No.: US 2019/0307474 A1
(43) Pub. Date: Oct. 10, 2019

(54) ROBOTICALLY SUPPORTED LAPAROSCOPIC ACCESS TOOLS

(71) Applicant: Maciej J. Kieturakis, Los Altos Hills, CA (US)

(72) Inventor: Maciej J. Kieturakis, Los Altos Hills, CA (US)

(21) Appl. No.: 16/378,087

(22) Filed: Apr. 8, 2019

Related U.S. Application Data
(60) Provisional application No. 62/655,662, filed on Apr. 10, 2018.

(51) Int. Cl. A61B 17/29; A61B 34/30

(52) U.S. Cl. CPC A61B 17/29; A61B 2017/00477; A61B 34/30

(57) ABSTRACT

Surgical tools intended for use with robotic system include a shaft having a distal effector end and a proximal attachment end. A flexible cable having a distal effector end and a proximal attachment end is slidably received in the central passage of the shaft, and a flexible cable wire assembly includes a pull/push wire having a distal effector end and a proximal attachment end slidably received in a lumen of the pull/push cable. An end effector is operably attached to the distal effector ends of the flexible cable and the distal effector end of the pull/push wire, and the end effector is disposed distally beyond the distal effector end of the shaft and is actuated by axially translating the pull/push wire relative to the flexible cable in the flexible cable wire assembly.

### CROSS-REFERENCE TO RELATED APPLICATIONS

The present application claims the benefit of Provisional Application No. 62/655,662 (Attorney Docket No. 41628-714.101), filed on Apr. 10, 2018, the full disclosure of which is incorporated herein by reference.

### BACKGROUND OF THE INVENTION

### 1. Field of the Invention

The present invention relates generally to medical systems, tools, and methods. More particularly, the present invention relates to systems and tools for robotically assisted laparoscopic access, typically for access of multiple robotically manipulated tools through a single incision in the umbilicus or other location.

In recent years, many open surgical procedures performed in the abdominal cavity have been replaced by minimally invasive procedures performed through several very small incisions using an endoscope, referred to as a laparoscope, inserted through one of the incisions. The other incisions are used for introducing surgical tools, and the abdominal cavity is inflated to create a space for performing the surgery. Such procedures are commonly called “laparoscopic”, and can be used for gallbladder removal, hernia repair, hysterectomy, appendectomy, gastric fundoplication, and other procedures. Similar endoscopic, thoracoscopic and other procedures are performed in other body cavities with or without inflation.

While a great advance over open surgical procedures, which can require an incision of several inches or more through the abdominal wall, such laparoscopic procedures still require incisions through muscle or fascia in several separate sites. Each incision may increase the risk of infection, bleeding trocar site hernia, increased postoperative pain, compromised cosmetic result and other adverse events for the patient.

As an improvement over such laparoscopic procedures, “single port” laparoscopy has been proposed where a single access port is inserted through the umbilicus (the patient's navel). Access solely through the umbilicus is advantageous since it provides superior cosmetic and functional results. Introducing the laparoscope and all other tools necessary for the surgery through a single port, however, makes performance of the procedures more difficult. In particular, the use of conventional laparoscopic tools, which are typically straight, makes it difficult to approach a single target area in the treated tissue with two or more tools at the same time.

Further improvements in the field of single port laparoscopic surgery are described in U.S. Patent Publications 2012/0116362 and 2016/0081752, commonly assigned with the present application, the full disclosures of which are incorporated herein by reference. As generally described in these applications, systems for performing single port laparoscopic procedures include a transcutaneous seal and a plurality of tools. The tools comprise a substantially rigid tubular seal having a core which is translatably and rotatably disposed in the sleeve. The handle at the proximal end of the tool controls an end effector at the distal end of the tool. The sleeves of the tools are locked in the transcutaneous seals and remain in a fixed geometric relationship which prevents the tools from interfering with each other during laparoscopic procedures. Adjacent tools are held by a pivot structure in US2012/0116362 and by an external frame having a double pivot arm in US2016/0081752. While functional and a significant advance in the art, it would be desirable to incorporate certain design features of such manual single port access systems into robotically manipulated laparoscopic tool manipulation systems.

Thus, it would be a benefit to provide further improved systems and tools for laparoscopic access through single and individual ports for performing minimally invasive robotic surgical procedures. It would be particularly desirable if the robotically manipulated tools were configured to permit multiple tool access to abdominal and other surgical target sites through small single incisions or the umbilicus with a minimum interference between adjacent tools during the performance of a procedure. At least some of these objectives will be met by the inventions described hereinafter.

### 2. Description of the Background Art

U.S. Patent Publications 2012/0116362 and 2016/0081752 have been described above. Surgical robotic systems of the type suitable for use with the laparoscopic tools of the present invention are described in US20060167440; US20090163931; US20140188130; US20110118709; US20130116712; US20160235496; US20070021738; and US20030045778.

### SUMMARY OF THE INVENTION

In a first aspect of the present invention, a laparoscopic tool is configured to be mounted on an arm of a surgical robotic system, such as a commercially available surgical robotic system of the type manufactured by Intuitive Surgical Systems, Inc., or any other commercial vendor. In particular, the laparoscopic tools of the present invention will be configured to be removably attached to the manipulable surgical robotic arms of the surgical robotic systems so that the robotic arms may freely move the laparoscopic tools in space, typically with six degrees of freedom, as well as manipulate components within the laparoscopic tools in order to move and actuate surgical effectors carried by the tools.

The tools described in this application are designed to work with the robotic systems, such as those which operate on the “remote center” principle. The robotic arms of such robots move in two planes rotated about the pivots which are positioned at the right angle to each other. A robotic cannula may be rotated by the arm in these two planes, e.g. up and down as well as laterally and medially or combination of these movements. These movements will change the position (angle) of the cannula in relation to the wall of the cavity maintaining the “remote center” of the cannula always in the point of insertion.

The laparoscopic tools of the present invention typically comprise a shaft, a flexible cable, a pull/push wire, and an end effector. The shaft has a distal effector end and a proximal attachment end. A central passage extends through the shaft between the ends and slidably receives the flexible cable which also has a distal effector end and a proximal attachment end. The pull/push wire, which also has a distal effector end and a proximal attachment end, is in turn slidably received in a lumen of the flexible cable to form a flexible cable wire assembly. The end effector is operably attached to both the distal effector end of the flexible cable and the distal effector end of the pull/push wire. The end effector will be disposed distally beyond the distal effector end of the shaft and may be actuated by axially translating the pull/push wire relative to the flexible cable wire assembly. In this way, the distal effector ends of each of the tool shaft, the flexible cable, and the pull/push wire are configured to be removably attached to the robotic arm of the surgical robotic system so that, in addition to moving the laparoscopic tool as a whole through free space, the robotic arm can also axially reposition the flexible cable wire assembly relative to the shaft, rotate the flexible cable wire assembly relative to the shaft, and axially translate the pull/push wire relative to the flexible cable of the flexible cable wire assembly to actuate the end effector.

In specific embodiments, the laparoscopic tool may further comprise a telescoping section extending distally of the distal effector end of the shaft. Such as telescoping section can accommodate extension and retraction of the flexible cable wire assembly which results from manipulation by the surgical robotic arm, while serving to support and protect the flexible cable wire assembly as it is being extended and retracted. The shaft of the laparoscopic tool may also further comprise (a) a semicircular mid-portion end (b) straight proximal and distal sections which lay along a common axis, where the flexible cable wire assembly can bend to accommodate the semicircular mid-portion as the flexible wire cable assembly is axially translated in the central passage of the shaft by the robotic arm.

In still further specific aspects, the distal end effector of the tool shaft may be configured to be attached to a robotic arm so that the robotic arm can reposition the entire tool with six degrees of motion. The flexible cable wire assembly may be configured to be rotatably and translatably attached to one or more driver(s) in the robotic arm so that these drivers can axially and rotationally reposition the flexible cable wire assembly relative to the shaft. Similarly, the pull/push wire will be configured to be translatably attached to one or more driver(s) in the arm to axially translate the pull/push wire relative to the flexible cable in the flexible cable wire assembly which in turn will actuate the end effector. In this configuration, the robotic arm and the tool are positioned in a configuration where an imaginary line connecting proximal and distal segments of the tool penetrates the cavity wall at the point of “remote center”. The distance from this point of remote center to the point of true insertion would be equal to the radius of the semicircle of a mid-segment of the tool

In a second aspect of the present invention, a method for performing robotic surgery utilizes two or more laparoscopic or other tools which are configured to pass through a single percutaneous port or other passage in a patient, typically a laparoscopic or other minimally invasive port, and often a port configured to provide access to the abdomen through the umbilicus. The method comprises providing a surgical robotic system of the type having at least first and second robotic arms. At least first and second surgical tools are provided, where each tool includes a shaft, a flexible cable and wire assembly, and an end effector, generally as described above. The first tool is attached to the first robotic arm, and the second tool is attached to the second robotic arm. The first and second robotic arms may be individually and/or simultaneously manipulated to position the shafts of the tools in free space and through the port or other passage as well as to operate the end effectors on each of the surgical tools to surgically interact with tissue while a mid-portion of each shaft remains positioned within the port in a manner such that the mid-portions of the shafts avoid interference during all or at least a portion of the surgical procedure.

In exemplary embodiments of the methods of the present invention, the mid-portions of each tool are semi-circular and extend radially inwardly from a common axis of proximal and distal sections of the shaft. Such curved geometries allow the first and second arms to be manipulated to rotate the tools about a virtual center point of the semi-circular mid-portion while that semi-circular mid-portion remains within the single percutaneous passage. Such geometries are particularly beneficial since they allow the surgical tools to be triangulated at the target while avoid interference of the shafts above the surgical site.

In other preferred aspects of the methods, the distal portion of at least some of the tools will provide telescoping sections extending distally of the distal end effector to accommodate extension and retraction of the flexible cable wire assembly.

In still further specific aspects of the methods of the present invention, the first and second robotic arms are manipulated to cause each of the (a) repositioning the entire tool with six degrees of motion, (b) rotating and translating the flexible cable wire assembly to axially and rotationally reposition the flexible cable wire assembly relative to the shaft, (c) axially translate the pull/push wire relative to the flexible cable in the flexible cable wire assembly to actuate the end effector.

In further aspects, the flexible cable wire assemblies of the present invention may further include a bidirectional torque tube located coaxially over the flexible cable and having a proximal end coupled to the one or more driver(s) in the robotic arm. The bidirectional torque tube will typically be configured to transmit both torque (rotation about its axis) and axial translation (translation along its axis) from the one or more driver(s) in the robot arm to the end effector. Use of the bidirectional torque tube in addition to the flexible cable of the flexible assembly is advantageous as the torque tube can be made from a more robust material such as a braid or other reinforced polymer tube, a counterwound coiled tube, or the like, in order to enhance both the transmission of axial and rotational forces.

In still further exemplary and preferred embodiments, the flexible cable wire assembly may further comprise an angulation disc and an angulation cord coupled to the one or more driver(s). Use of the angulation disc and the angulation cord allows the end effector to be rotated about an axis normal to the axial direction of the bidirectional torque tube. In particular, such structure allows the end effector, which is typically an asymmetric jaw structure or similar asymmetric effector, to be rotated about an axis transverse to the shaft while maintaining the position of all other shaft components immobile.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a commercially available robotic surgical system of the type that can be used to manipulate the laparoscopic tools of the present invention.

FIG. 2 illustrates a pair of laparoscopic tools intended for manual manipulation in surgical procedures where said tools are intended for single port access and are pivotally mounted in a support frame, with a repositioned view of one of the tools shown in broken line.

FIGS. 3A-3D illustrate a laparoscopic tool constructed in accordance with the principles of the present invention with FIGS. 3A and 3B showing the tool with an extended and a retracted telescopic distal extension, respectively, and FIGS. 3C and 3D showing internal component parts of the laparoscopic tool.

FIG. 4 illustrates mounting of a proximal attachment end of the laparoscopic tool onto an arm of a surgical robot.

FIG. 5 illustrates three laparoscopic tools constructed in accordance with the principles of the present invention attached to three arms (shown partially) of a surgical robotic system (not shown) performing a single port laparoscopic surgical procedure.

FIGS. 6 and 7 illustrate alternative attachments of the laparoscopic tools to the surgical robotic arms.

FIG. 8 illustrates the incorporation of a bidirectional torque cable coaxially over a Bowden cable with an end effector angulation cord between the bidirectional torque cable and the Bowden cable in a particular embodiment of the laparoscopic tool of the present invention.

FIG. 9 is a cross-sectional view of a distal portion of a laparoscopic tool of the present invention showing connections of the bidirectional torque cable, the Bowden cable, and the end effector angulation cord of FIG. 8 to an end effector.

FIGS. 10A through 10C illustrate how the bidirectional torque cable, the Bowden cable, and the end effector angulation cord of FIGS. 8 and 9 are used to manipulate the end effector in accordance with the present invention.

FIG. 11 illustrates an alternative embodiment of an end effector mounted on a steerable shaft segment.

FIG. 12 is a detailed view of the steerable shaft segment of FIG. 11 with portions broken away.

### DETAILED DESCRIPTION OF THE INVENTION

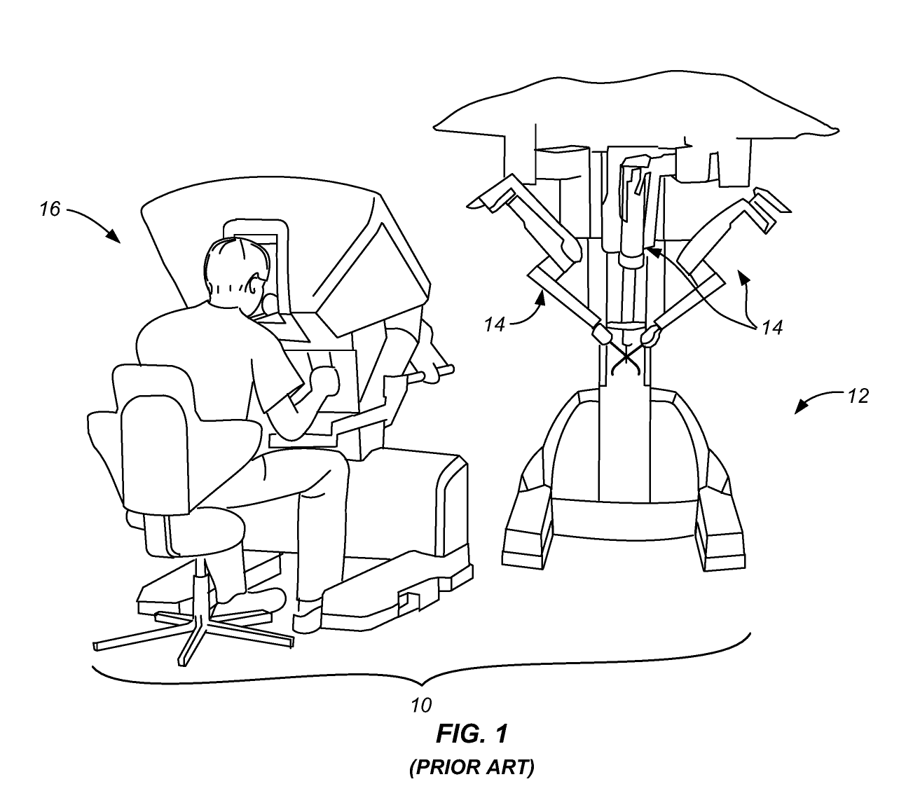

Referring now to FIG. 1, the laparoscopic tools and end effectors of the present invention are intended to be used with and manipulated by known and commercially available robotic systems, such as a da Vinci® Surgical System available from Intuitive Surgical, Inc., Sunnyvale, Calif. An exemplary robotic surgery system 10 includes a robotic station 12 that includes a plurality of robotic arms 14 (with three being illustrated) and a controller module 16 where a physician can view the procedure and control the surgical arms to manipulate the tools to perform a desired laparoscopic or other surgery.

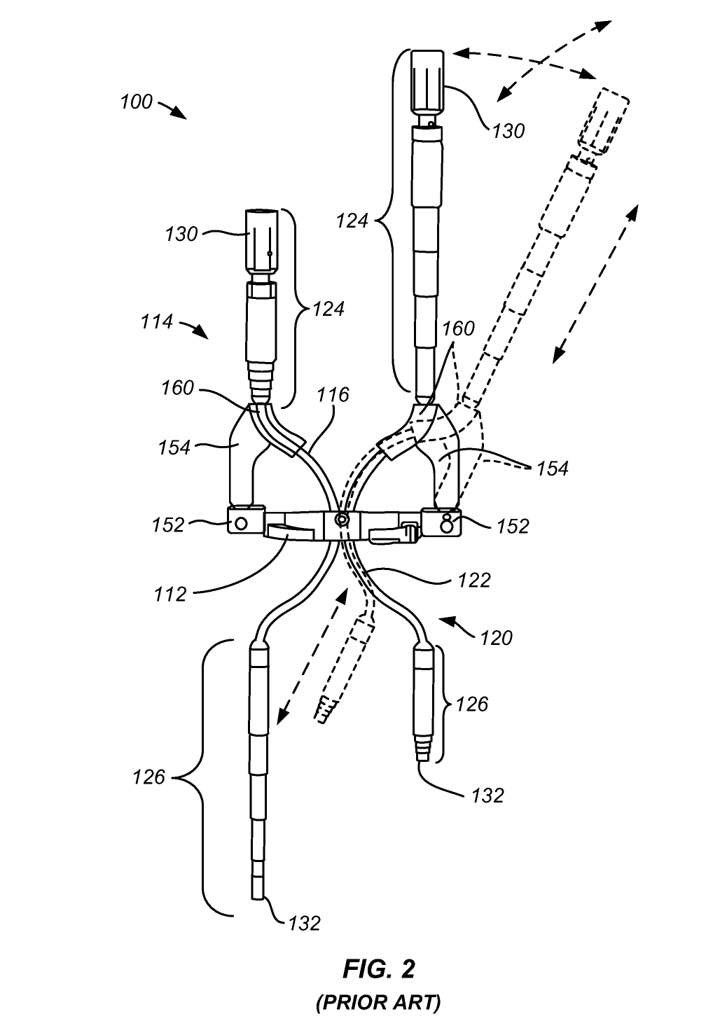

Referring now to FIGS. 2, a prior art laparoscopic tool system 100 of the type described in US2016/0081752, previously incorporated herein by reference, comprises a tool attachment frame 112 having a first tool 114 and a second tool 120 pivotally attached thereto. The first tool has a mid-portion 116 and the second tool has a mid-portion 122, and both mid-portions extend generally inwardly from an axis 128 of the tool. Both mid-portions 116 and 122 are preferably circular and have a radius emanating from a virtual rotation point which is generally aligned with a pivot 152 of an assembly attached to an outer periphery of the tool attachment frame 112. Having the virtual rotation points of each tool located outside the periphery of the ring in the location of double pivot allows the generally circular midportions 116 and 22 to pass and move through the central opening 118 of the frame 112 without interfering with each other. While the mid-portions 116 and 22 could alternatively have non-circular geometries which extend radially inward relative to the frame 112, for example being oval or polyhedral, the circular shape causes the passage point of the mid-portion to remain fixed within the central opening 118 of the frame so long as the tool is constrained to move in to orthogonal planes by the pivot attachment as will be explained in more detail hereinafter. While in some instances, it would be possible to modify the arms of a surgical robot to manipulate these prior art tools, these tools are intended to be manually manipulated and any attempt to directly interface the look with a robotic arm would be suboptimum.

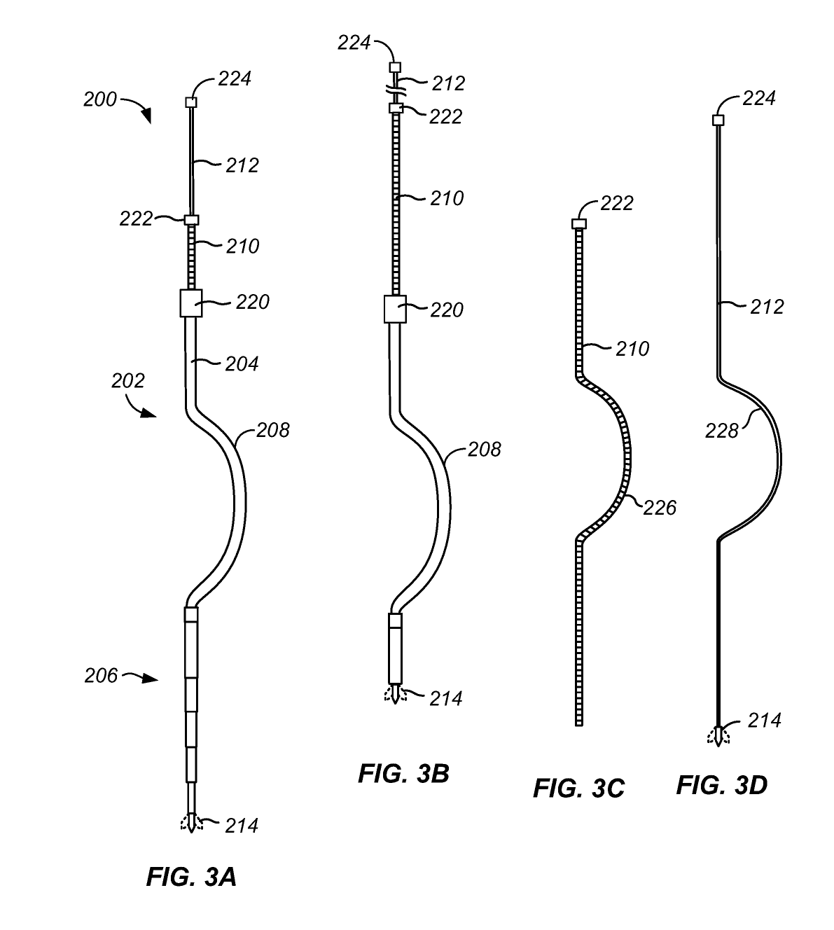

Referring now to FIGS. 3A-3D, a laparoscopic tool 200 constructed in accordance with the principles of the present invention comprises a shaft 202 having a proximal section 204 and a distal section 206 separated by a mid-portion 208. The shaft has a hollow central passage which receives a flexible cable 210. The flexible cable 210 has a hollow lumen extending from a distal end to a proximal end thereof which receives a pull/push wire 212 having an end effector 214 at its distal end. The shaft 202 has a proximal attachment member 220 at its distal end. The flexible cable 210 has a proximal attachment member 222 at its proximal end, and the pull/push wire 212 has a proximal attachment member 224 at its proximal end. The distal section 206 of the shaft 202 is preferably joined as a telescoping structure having a plurality of segments 216 including a distal-most segment 218 that carries the end effector 214. The telescoping distal section may be axially extended and retracted to accommodate full axial extension of the flexible cable 210, as illustrated in FIG. 3A, as well as full axial retraction of the flexible cable, as illustrated in FIG. 3B. The flexible cable 210, by nature of its flexibility, provides a conformable central region 226 to accommodate bending as the cable passes through a preferred C-shaped mid-portion 208 of the shaft. Similarly, the pull/push wire 212 will have a conforming region 228 to accommodate bending as it is extended and retracted through the conforming region 226 of the flexible cable 210.

The laparoscopic tool 200 can thus allow manipulation of the end effector 214 using a surgical robotic arm in a number of ways. First, the entire laparoscopic tool 200 can be moved through free space, typically with up to six degrees of freedom, by grasping and moving the proximal attachment member 220 of the shaft. By “six degrees of freedom of movement, it is meant that the surgical arm can move the arm (1) forward/backward along the tool's axis, (2) laterally (side-to-side) in a first direction orthogonal to the axis, (3) laterally (side-to-side) in a second direction orthogonal to the axis and to the first direction, and (4-6) rotation about each of the three perpendicular axes, i.e. yaw (first lateral axis), pitch (second lateral axis), and roll (longitudinal tool axis).

In specific embodiments, the robot arm will move the laparoscopic tool 200 in at least three different directions including up and down (i.e. closer to the patient and away from). Such up and down movement may be used at the beginning of the procedure for example during the setup. Once the set-up is complete, the distance from a proximal portion of the laparoscopic tool held by the robotic arm to the single port or other entry point into the body cavity will typically remain the same. The initial distance is selected so a “remote center” 208 (FIGS. 7 and 8) of the laparoscopic tool 200 is at the level of the entry point into the body cavity. From this time on, all movement of the robotic arm will maintain this distance so that the remote center remains the same location at the point of entry through the abdominal wall.

Axial translation of the cable and wire assembly (including the flexible cable 210 and pull/push wire 212) relative to the shaft 202 can be achieved by grasping and manipulation of the proximal attachment member 222 at the proximal end of the flexible cable 210. Similarly, rotation of the cable and wire assembly about the assembly's longitudinal axis can also be achieved by grasping and rotation of the proximal attachment member 222 at the proximal end of the flexible cable 210. In addition, axial translation of the pull/push wire 212 relative to the flexible cable 210 to actuate an end effector may be achieved by manipulation of the proximal attachment 224 at the proximal end of the pull/push wire 212.

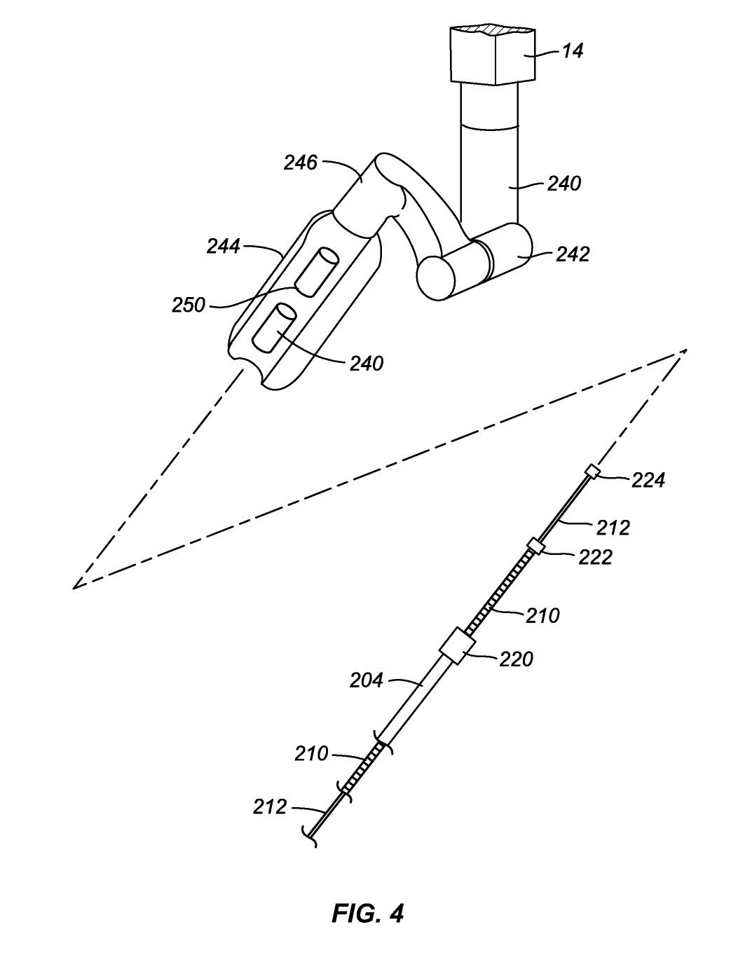

Referring now to FIG. 4, such manipulation of the proximal attachments members 220, 222, and 224 by a surgical arm 14 is schematically illustrated. The surgical arm 14 may include a first arm segment 240 and a second arm segment 246 connected to the first arm segment by a rotatable joint 242. A tool connector 244 having at least a first driver 248 and a second driver 250 is connected to the distal or free end on the second arm segment 246. The laparoscopic tool 200 may be attached to the tool connector 244 and operably coupled to the drivers 248 and 250 by connecting the proximal attachment member 220 of the shaft directly to the tool connector 244. The proximal attachment member 222 of the flexible cable may be connected to the first driver 248 while the proximal attachment member 224 of the pull/push wire may be connected to the second driver 250 herein.

The first driver 248 will typically be configured to both axially translate and rotate the proximal attachment member 222 on the flexible cable 210 while the second driver 250 will typically be configured to at least axially translate and optionally rotate the proximal attachment member 224 on the pull/push wire 212. In this way, the laparoscopic tool 200 can be attached to and be fully manipulated via the surgical arm 14 of the surgical robot during a surgical procedure.

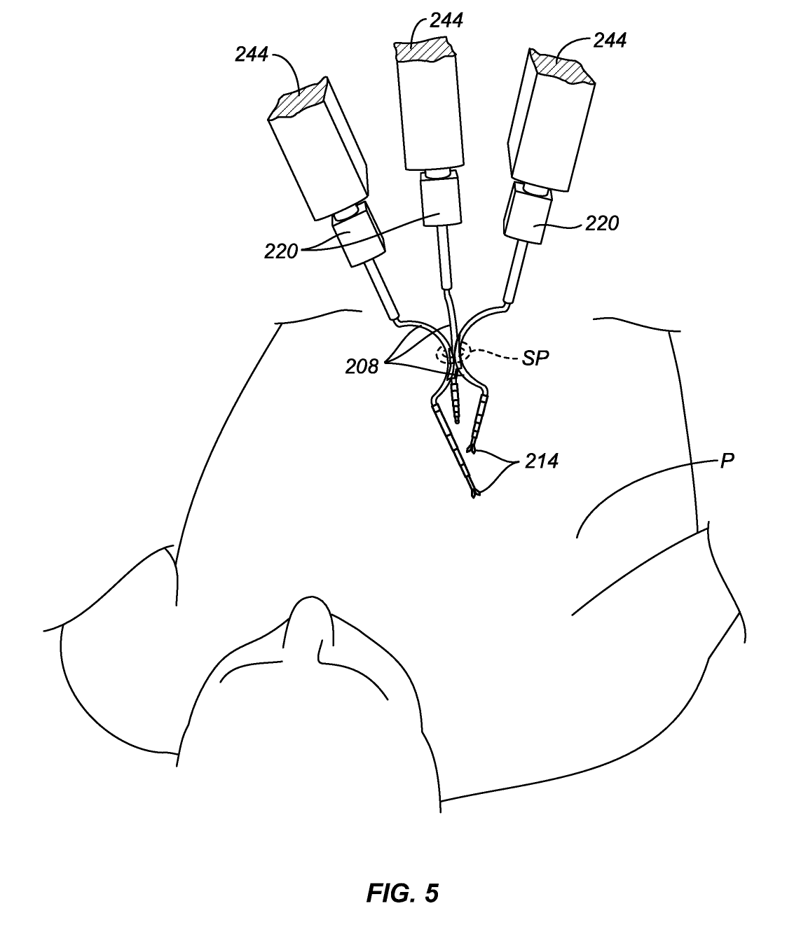

Referring now to FIG. 5, the tool connectors 244 of the three surgical robotic arms 14, as illustrated previously in FIG. 1, can be attached to the proximal attachment members 220 of three different laparoscopic tools as generally described previously. Instead of an end effector, however, one of the surgical tools may provide a laparoscope to allow viewing of the surgical site during performance of the procedure. The three laparoscopic tools 200 are typically introduced sequentially through a single port (SP) such that the C-shaped mid-portions 208 of each are clustered within the access passage provided by the port. The robotic arms 14 are able to move each of the laparoscopic tools 200 independently so that the C-shaped mid-portions 208 remain within the single port SP with minimal or no interference with each other. The end effectors 214, however, can remain laterally separated as they are engaged with tissue to perform the desired procedure. Specific movements of the end effectors will be provided by manipulating the flexible cable and pull/push wires separately using the internal drivers shown in FIG. 4.

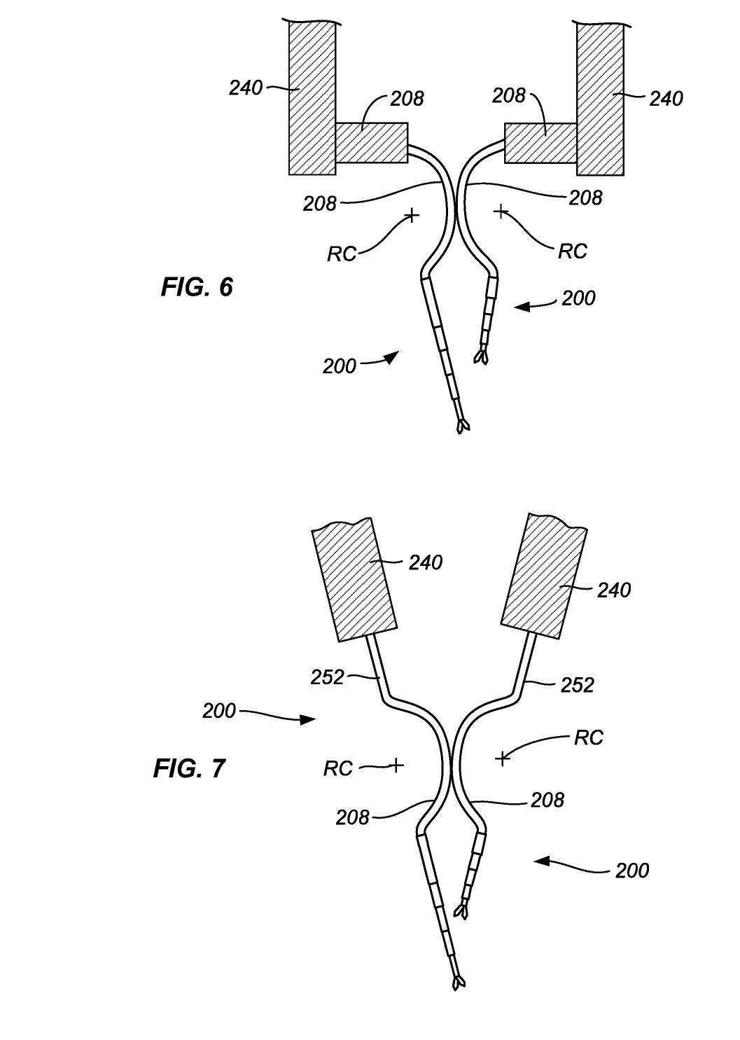

FIGS. 6 and 7 illustrate alternative attachments of the laparoscopic tools 200 to the surgical robotic arms. In particular, FIG. 6 illustrates the C-shaped mid-portions 208 of the tools being attached by lateral extensions from the bottoms of each arm segment 240. FIG. 7 illustrates straight proximal shaft portions 252 extending into the bottoms of each surgical arm segment 240.

FIG. 8 illustrates an optional modification to the flexible cable assembly where a bidirectional torque tube 256 is located coaxially over the exterior of the flexible cable 210. An end effector angulation cord 258 extends through an annular gap between the interior surface of the bidirectional torque tube 256 and an exterior surface of the flexible cable 210. The pull wire 212 for actuating the end effector 214 is disposed in a central lumen of the flexible cable 210, as in prior embodiments.

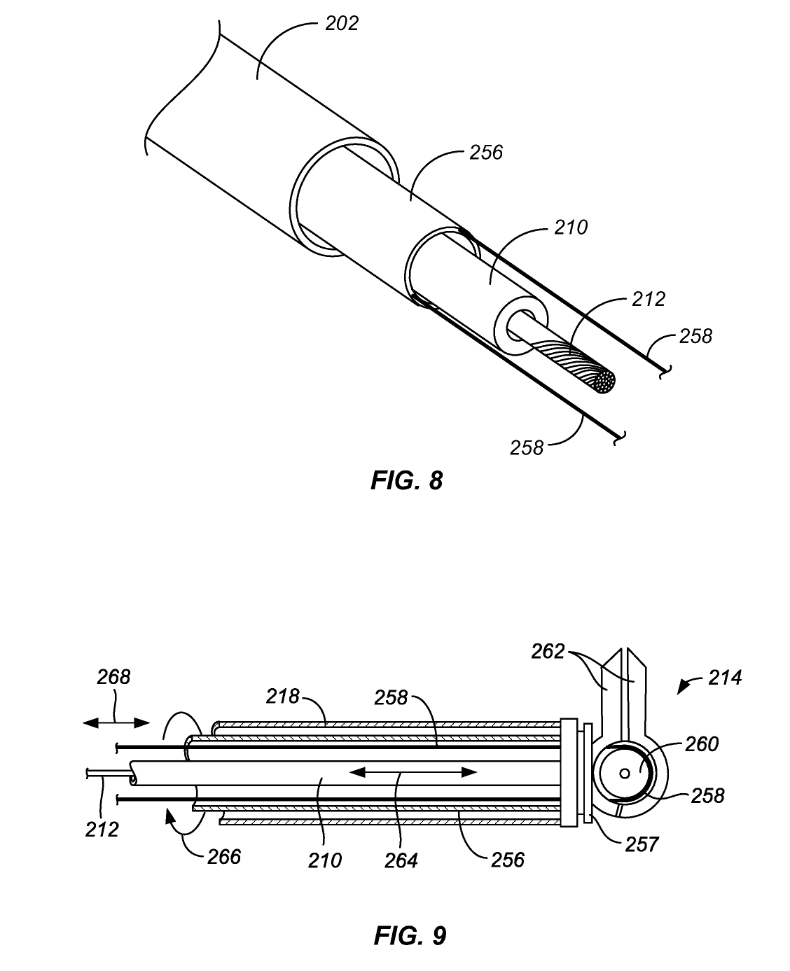

Referring now to FIG. 9, the assembly of the flexible tube 210, the pull wire 212, the bidirectional torque tube 256, and the end effector angulation cord 258 extends through a distal-most segment 218 of the telescoping distal section 206 of the device shaft 202. The bidirectional torque tube 256 is typically attached to a rotating base 257 so that rotation of the torque tube will rotate the end effector 214 which is mounted on the base. The torque tube 256 may be rotated together with the flexible tube 210 in the same manner as the flexible tube is rotated by itself. The addition of the torque tube enhances the torsional stiffness as well as the axial pushability of the flexible tube assembly. Thus, pulling and pushing the torque tube 256 in the directions of arrow 264. In addition, rotation of the torque tube 256 in the directions of arrow 266 will cause the end effector 214 to rotate about an axis of the flexible tube assembly and the distal-most segment of the shaft.

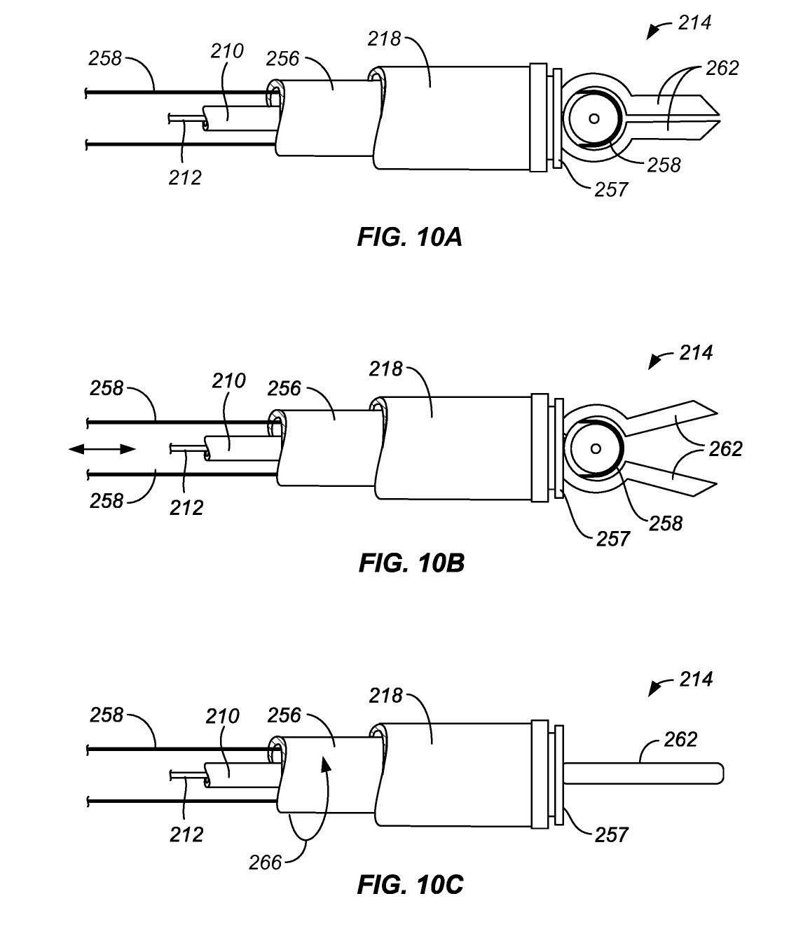

In addition, the end effector 214 may be rotated about an axis transverse to the axis of the shaft segment 218 by drawing on either end of the end effector angulation cord 258, as shown by arrow 268. The end effector angulation cord 258 is disposed around the periphery of an angulation disc 260 which in turn is coupled to the end effector 214. The end effector angulation cord will cause the angulation disc 260 to turn and cause the end effector to turn as well. The annular space between the exterior of the flexible tube 210 and the inner wall of the bidirectional torque tube 256 also protects the end effector angulation cord 258, and additional eyelets, channels, and other structure may be provided in the annular space to assure that the end effector angulation cord 258 can be pulled in either direction to rotate the end effector, for example from a laterally deflected orientation as shown in FIG. 9 to an axially aligned orientation as shown in FIG. 10A.

Referring now to FIGS. 10A through 10C, use of the flexible cable assembly for manipulating the end effector 214 will be described. As shown in FIG. 10A, the end effector 214 may comprise a pair of jaws pivotally mounted on a base 257 to open (FIG. 10B) and close (FIGS. 9 and 10A). Initially, the jaws 262 may be closed, as shown in FIG. 10A. The jaws may then be opened, as shown in FIG. 10B, by drawing on the pull wire 212 in the lumen of the flexible cable 210, as described previously. The jaws or other end effectors may be rotated of deflected about an axis transverse to the longitudinal of axis of the distal-most shaft segment 218, typically over ±180°, by causing a driver in the robot arm to draw on either end of the end effector angulation cord 258. As shown in FIG. 10C, the end effector 214 may be further rotated about the longitudinal axis of the distal-most segment 218 of the device shaft by rotating the bidirectional torque tube 256 in the direction of arrow 266.

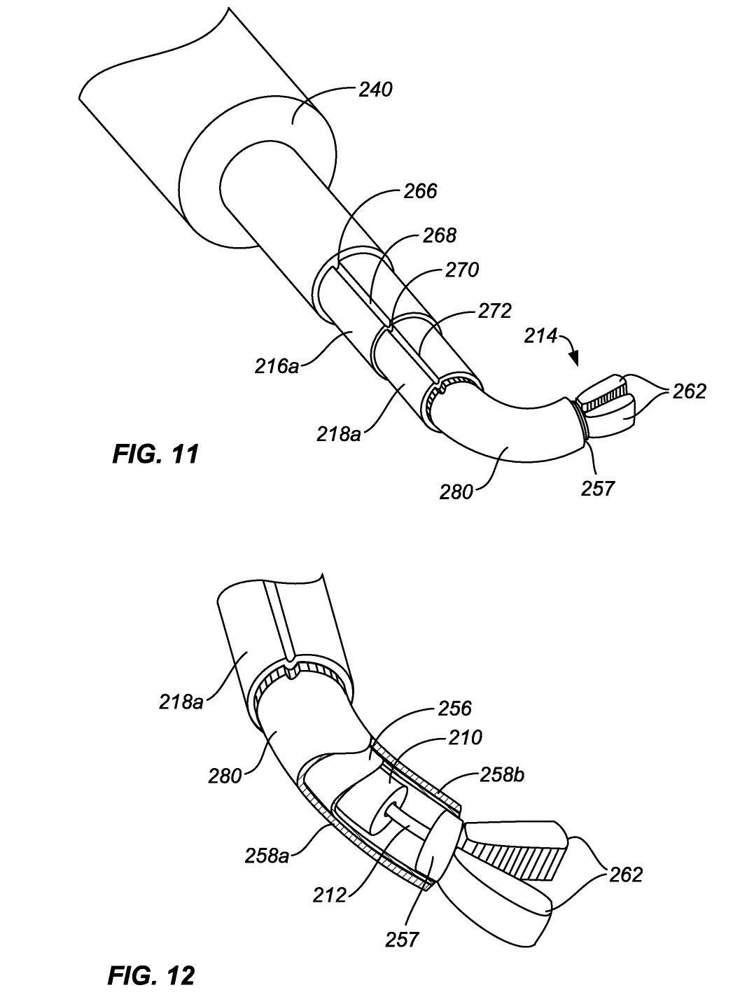

Referring now to FIGS. 11 and 12, an alternative embodiment of an end effector assembly comprising a steerable shaft segment 280 is described. The steerable shaft segment 280 may comprise any type of flexible sleeve as is commonly known in the catheter and laparoscopic tool art, including reinforced and non-reinforced polymer tube, tubes formed from a plurality of ring segments, slotted metal and polymeric tubes, and the like. The steerable shaft segment 280 typically extends distally from a data-most shaft segment 218a, and the angulation cord 258 will extend through lumens or other passages formed in a wall of the steerable shaft segment. The angulation cord will typically be formed in two separate lengths 258a and 258b so that tension on one of the lengths will bend the steerable segment in a first direction and tension on the other of the lengths will bend the segment in an opposed second direction. As shown in FIG. 12, tension is applied to length 258b, causing the steerable shaft segment to bend toward the right as illustrated.

In order to accommodate a twisting torque that may applied to the steerable shaft segment, the telescoping segments 216 and 218 segments of the tool shaft 202 will be provided with alignment features to prevent rotational misalignment. For example, each of the segments 216a and 218a as illustrated in FIGS. 11 and 12 may be provided with pins or tracks 266 and 270 which can slide in exterior grooves 268 and 272 as he segments telescope in and out to prevent relative rotation.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A laparoscopic tool for mounting on an arm of a surgical robotic system, said laparoscopic tool comprising:
a shaft having a distal effector end and a proximal attachment end, said shaft having a central passage extending between said ends;
a flexible cable wire assembly slidably received in the central passage of the shaft, said flexible cable wire assembly comprising a flexible cable having a distal effector end and a proximal attachment end and a pull/push wire having a distal effector end and a proximal attachment end slidably received in a lumen of the flexible cable; and
an end effector operably attached to the distal effector end of the flexible cable and the distal effector end of the pull/push wire, wherein the end effector is disposed distally beyond the distal effector end of the shaft and is actuated by axially translating the pull/push wire relative to the flexible cable in the flexible cable wire assembly;
wherein the distal effector ends of each of the tool shaft, the flexible cable, and the pull/push wire are configured to be removably attached to the arm of the surgical robotic system so that the arm can at least axially reposition the flexible cable wire assembly relative to the shaft, rotate the flexible cable wire assembly relative to the shaft, and axially translate the pull/push wire relative to the flexible cable of the flexible cable wire assembly to actuate the end effector.

2. A laparoscopic tool as in claim 1, further comprising a telescoping section extending distally of the distal effector end of the shaft to accommodate extension and retraction of the flexible cable wire assembly.

3. A laparoscopic tool as in claim 1, whirring segments of the telescoping section have alignment features that prevent relative rotation as the segments are extended and retracted.

4. A laparoscopic tool as in claim 1, wherein the shaft comprises (a) a semicircular mid-portion and (b) straight proximal and distal sections which lay along a common axis, wherein the flexible cable wire assembly bends to accommodate the semicircular mid-portion as the flexible cable wire assembly is axially translated in the central passage of the shaft by the robot arm.

5. A laparoscopic tool as in claim 1, wherein (a) the distal effector end of the tool shaft is configured to be attached the robotic arm so that the robot arm can reposition the entire tool with six degrees of motion, (b) the flexible cable wire assembly is configured to be rotatably and translatably attached to one or more driver(s) in the robot arm so that the driver(s) can axially and rotationally reposition the flexible cable wire assembly relative to the shaft, and (c) the pull/push wire is configured to be translatably attached to one or more drivers in the arm to axially translate the pull/push wire relative to the flexible cable in the flexible cable wire assembly to actuate the end effector.

6. A laparoscopic tool as in claim 1, wherein the flexible cable wire assembly further comprises a bidirectional torque tube located coaxially over the flexible cable and having a proximal end coupled to the one or more driver(s) in the robot arm, wherein the bidirectional torque tube is configured to transmit torque and axial translation forces from the one or more driver(s) in the robot arm to the end effector.

7. A laparoscopic tool as in claim 5, wherein the flexible cable wire assembly further comprises an angulation disc and an angulation cord coupled to the one or more driver(s) to rotate the end effector about an axis normal to a central axis of the bidirectional torque tube.

8. A laparoscopic tool as in claim 1, wherein the shaft includes a steerable end segment at the distal effector end.

9. A method for performing robotic surgery with at least two tools passing through a single percutaneous passage, said method comprising:
providing a surgical robotic system having at least first and second robotic arms;
providing at least first and second surgical tools, wherein each tool includes:
(i) a shaft having a distal effector end and a proximal attachment end, said shaft having a central passage extending between said ends;
(ii) a flexible cable wire assembly slidably received in the central passage of the shaft, said flexible cable wire assembly comprising a flexible cable having a distal effector end and a proximal attachment end and a pull/push wire having a distal effector end and a proximal attachment end slidably received in a lumen of the flexible cable; and
(iii) an end effector operably attached to the distal effector end of the flexible cable and the distal effector end of the pull/push wire, wherein the end effector is disposed distally beyond the distal effector end of the shaft and is actuated by axially translating the pull/push wire relative to the flexible cable in the flexible cable wire assembly;
attaching the first tool to the first robotic arm;
attaching the second tool to the second robotic arm;
manipulating the first and second arms to operate the end effectors to surgically interact with tissue while a mid-portion of each shaft is positioned in the single percutaneous passage and said mid-portions avoid interference.

10. A method as in claim 9, wherein the mid-portions of each tool are semi-circular and extend radially inwardly from a common axis of proximal and distal sections of the shaft and wherein the first and second arms are manipulated by the surgical robotic arms to rotate each of the tools about a virtual center point of the semi-circular mid-portion while the semi-circular mid-portion remains with the single percutaneous passage.

11. A method as in claim 9, wherein each tool comprises a telescoping section extending distally of the distal effector end of the shaft to accommodate extension and retraction of the flexible cable wire assembly.

12. A method as in claim 9, wherein manipulating the first and second robot arms effects each of (a) repositioning the entire tool with six degrees of motion, (b) rotating and translating the flexible cable wire assembly to axially and rotationally reposition the flexible cable wire assembly relative to the shaft, and (c) axially translating the pull/push wire relative to the flexible cable in the flexible cable wire assembly to actuate the end effector.

13. A method as in claim 9, wherein the flexible cable wire assembly further comprises a bidirectional torque tube located coaxially over the flexible cable and having a proximal end coupled to the one or more driver(s) in the robot arm, wherein the bidirectional torque tube is configured to transmit torque and axial translation forces from the one or more driver(s) in the robot arm to the end effector.

14. A method as in claim 13, wherein the flexible cable wire assembly further comprises an angulation disc and an angulation cord coupled to the one or more driver(s) to rotate the end effector about an axis normal to a central axis of the bidirectional torque tube.

15. A method as in claim 13, further comprising tensioning an angulation wire to bend a steerable end segment on the shaft to laterally deflect the end effector.
